SEQUENCE LISTING (2 sequences)

SEQ ID NO: 1 - DNA, 1170 bp, Aeropyrum pernix K1
gtggccctcgccgacataagtggttacctggacgtcctcgactcagttagggggttttcctatctggaaaacgcgagggaggtcctgaggagcggagaggctagatgcctaggaaacccccgcagcgagccggagtatgttaaggcgctctacgttataggcgcgtccagaataccggtaggggatggttgcagccacactctagaagagctcggggtattcgatatcagcgtgcccggcgagatggtgttcccatcgcccctagacttctttgaaagggggaagcccacccccctggtgaggtcccgcctccagctgccaaacggcgtcagggtctggctgaagctcgaatggtacaatcccttcagcctcagcgttaaggataggcccgcggtggaaatcatatctaggctctcgaggagggtggagaagggctcgctggttgcagacgccacctcgtccaacttcggtgtagcactctcggccgtggcgaggctctacggctatagagcgcgggtctacctgcccggggctgcagaggagttcggaaagctcctccccaggcttctaggggctcaggttattgtagaccccgaggcgccatcgaccgttcacctcctacccagggtgatgaaggactccaagaacgaggggttcgttcacgtgaaccagttctacaacgacgctaacttcgaggcccacatgagggggactgcaagggagatattcgtccagtcgcgcaggggagggctagcccttaggggggtcgccgggagtctagggacgtcgggccatatgtcggcagcggctttctaccttcagagcgtcgacccaagcattagagctgtgttagtgcagcccgcacagggagattccatacctggtataaggagggttgagacgggcatgctctggataaacatgctcgacataagctataccctcgccgaggtaacgctggaggaggcgatggaggccgtcgtagaggtggccaggagcgacgggctagtcatcgggccctccggcggggcagcggtgaaggccctggctaagaaagcggcggaaggggatctggagccgggagactacgtggtcgtagtgccagacacggggttcaagtacctaagtctcgtgcagaatgcgctggaaggagccggagactcggtctag

SEQ ID NO: 2 - protein, 389 aa, Aeropyrum pernix K1
VALADISGYLDVLDSVRGFSYLENAREVLRSGEARCLGNPRSEPEYVKALYVIGASRIPVGDGCSHTLEELGVFDISVPGEMVFPSPLDFFERGKPTPLVRSRLQLPNGVRVWLKLEWYNPFSLSVKDRPAVEIISRLSRRVEKGSLVADATSSNFGVALSAVARLYGYRARVYLPGAAEEFGKLLPRLLGAQVIVDPEAPSTVHLLPRVMKDSKNEGFVHVNQFYNDANFEAHMRGTAREIFVQSRRGGLALRGVAGSLGTSGHMSAAAFYLQSVDPSIRAVLVQPAQGDSIPGIRRVETGMLWINMLDISYTLAEVTLEEAMEAVVEVARSDGLVIGPSGGAAVKALAKKAAEGDLEPGDYVVVVPDTGFKYLSLVQNALEGAGDSV

(19) United States
(12) Patent Application Publication
(10) Pub. No.: US 2004/0002075 A1
(43) Pub. Date: Jan. 1, 2004

(54) Heat-resistant cysteine synthase

(76) Inventors: Kazuhiko Ishikawa, Ikeda-shi (JP); Koshiki Mino, Ikeda-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/301,916

(22) Filed: Nov. 21, 2002

(30) Foreign Application Priority Data
Nov. 22, 2001 (JP) 2001-356895

(51) Int. Cl. C12Q 1/68

(52) U.S. Cl. 435/6

(57) ABSTRACT

The inventor isolated heat-resistant cysteine synthase having substantially no decline in activity after heat treatment at 80° C. for 6 hours from the hyperthermophilic archaea Aeropyrum pernix, and determined the amino acid sequence and base sequence. The DNA encoding this enzyme is DNA consisting of the base sequence set forth in SEQ ID NO: 1. The enzyme has the amino acid sequence set forth in SEQ ID NO: 2. The enzyme can be used to carry out an efficient enzyme reaction on highly concentrated substrates prepared at elevated temperatures.

### BACKGROUND OF THE INVENTION

1. Field of the Invention

The present invention relates to a heat-resistant cysteine synthase. DNA encoding the enzyme, a vector comprising the DNA, a transformant comprising the vector, and a method for producing heat-resistant cysteine synthase using the transformant.

2. Description of the Prior Art

In the prior art, sulfur-containing organic compounds have been synthesized mainly chemically. Because the chemical synthesis of sulfur-containing organic compounds results in the unavoidable production of impurities due to side-reactions, impurities need to be eliminated. Another problem is environmental pollution and the like due to by by-products such as sulfur oxides. Furthermore, a catalyst is often required, resulting in higher costs.

In order to overcome the above problems, a method for synthesizing sulfur-containing organic compounds using O-acetylserine sulfhydrylase, which is a cysteine synthase, has been proposed (Flint et al., J. Biol. Chem., 271(1996), 16053-16067). Previously known O-acetylserine sulfhydrylase can catalyze about a variety of reactions involving sulfur transfer. The biochemical synthesis of sulfur-containing organic compounds using cysteine synthase results in extremely few by-products, and thus lowers the purification costs, gives products of higher purity and also overcomes problems with environmental pollution. The biochemical synthesis lowers costs because it does not need any catalysts.

Examples of cysteine syntheses include enzymes derived from Methanosarcina thermophila, Citrullus vulgaris, Salmonella typhimuriumu, Escherichia coli, plants and the like.

However, hitherto known cysteine synthases exhibit low heat resistance, i.e., are heat labile. The solubility of a solute in water is generally raised with temperature. Accordingly, heat-resistant cysteine synthase capable of functioning at elevated temperature would allow target substances to be produced more efficiently through the action of the heat-resistant cysteine synthase on highly concentrated substrate solutions prepared at high temperatures.

### BRIEF SUMMARY OF THE INVENTION

A main object of the present invention is to provide a heat-resistant cysteine synthase capable of more efficient synthesis of amino acids, representatively including cysteine, DNA encoding the enzyme, a vector comprising the DNA, a transformant comprising the vector, and a method for producing heat-resistant cysteine synthase using the transformant.

To achieve the above object, the present inventors aimed at hyperthermophilic archaeabacteria or archaea capable of growing at the high temperatures of 90 to 100° C. Archaea are organisms belonging to a third group of organisms distinct from eucaryotes and prokaryotes. They are considered to be descended from primeval organisms, and are special organisms which have neither evolved nor adapted to ordinary temperature environments.

The present inventors were the first to isolate such hyperthermophilic archaea-derived cysteine synthase. They also found the activity of the enzyme is substantially unimpaired by heat-treatment at 80° C. for 6 hours.

The present invention was completed on the basis of the above findings, and provides the following:

1. Heat-resistant cysteine synthase of which enzymatic activity is substantially unimpaired by heat treatment at 80° C. for 6 hours.

2. Heat-resistant cysteine synthase according to Item 1, derived from the hyperthermophilic archaea Aeropyrum pernix.

3. Heat-resistant cysteine synthase with at least 80% residual activity after heat treatment at 100° C. for 6 hours.

4. Heat-resistant cysteine synthase according to Item 3, derived from the hyperthermophilic archaea Aeropyrum pernix.

5. A polypeptide of (1) or (2) below:

(1) a polypeptide comprising the amino acid sequence set forth in SEQ ID NO: 2.

(2) a polypeptide which comprises the amino acid sequence in SEQ ID NO: 2 but containing one or more deletions, substitutions or additions of amino acid residues, and has heat resistant cysteine synthase activity.

6. DNA of (3), (4), or (5) below:

(3) DNA comprising the base sequence set forth in SEQ ID NO: 1.

(4) DNA comprising DNA which hybrides under stringent conditions, with DNA consisting of the base sequence in SEQ ID NO: 1, and encodes a polypeptide having heat-resistant cysteine synthase activity.

(5) DNA encoding the polypeptide according to Item 5.

7. A vector comprising the DNA according to Item 6.

8. A transformant comprising the vector according to Item 7.

9. A method for producing heat-resistant cysteine synthase which comprises incubating the transformant according to Item 8, and recovering the cysteine synthase from the transformant.

The present invention provides cysteine synthase with excellent heat resistance. Accordingly, the use of the enzyme of the present invention allows highly concentrated substrate solution prepared at high temperature to be used to bring about a variety of efficient synthetic reactions. For example, from O-acetylserine and a nucleophile such as sulfide, the corresponding amino acid can be efficiently synthesized at elevated temperature and the harmful hydrogen sulfide contained in various processed products can be removed by efficiently converting it into harmless and greatly value-added amino acids at high temperature.

The cysteine synthase of the present invention is also stable at room temperature, and can thus withstand long-term storage.

Enzymes are generally liable to lose its activity in the presence of organic solvents, but the present enzyme is also stable against organic solvents. Reactions can therefore be carried out using the cysteine synthase of the invention in organic solvents or aqueous solution containing organic solvents. As a result, even substrates that are poorly soluble in aqueous solution can be used as the reaction substrate, resulting in a greater range of applicable reaction targets.

### BRIEF DESCRIPTION OF THE DRAWINGS

FIG. 1 is a graph showing the optimal pH of the cysteine synthase which is an example of the present invention;

FIG. 2 is a graph showing the optimal temperature of the cysteine synthase which is an example of the present invention; and

FIG. 3 is a graph showing the heat resistance of the cysteine synthase which is an example of the present invention.

### DETAILED DESCRIPTION OF THE INVENTION

The present invention is described in detail below.

(I) Heat-Resistant Cysteine Synthase of the Invention Heat Resistance

The heat-resistant cysteine synthase of the present invention is an enzyme with remarkably excellent heat resistance, wherein 6 hours of heat treatment at 100° C. results in substantially no decline in its activity. In the present invention, “substantially no decline in activity” or “activity is substantially unimpaired” includes, for example, retaining at least 90% activity. The enzymes of the invention include heat-resistant cysteine synthase with at least 80% residual activity after heat treatment at 80° C. for 6 hours. The maximum temperature at which the heat-resistant cysteine synthase of the invention exhibits activity is usually about 90° C.

The heat-resistant cysteine synthase of the invention is preferably an enzyme of which the optimal temperature when determining the initial rate of enzyme reaction is 60° C. or higher, although this varies depending on the kind of buffer in which the enzyme reaction is carried out.

The enzyme activity while estimating the above heat resistance is the value determined by the activity assaying method (1) described in the item of EXAMPLE. The enzyme activity while determining the optimal temperature is the value determined by the activity assaying method (2) described in the item of EXAMPLE.

Stability at Room Temperature

The heat-resistant cysteine synthase of the invention also has excellent stability at room temperature. It is preferable that the cysteine synthase of the present invention has 90% or more residual activity after incubation for 4 hours in 50 mM potassium phosphate buffer (pH 7.0) at 30° C. The enzyme activity is the value obtained by assaying the activity in synthesizing cysteine from O-acetylserine and sodium sulfide in 50 mM potassium phosphate buffer (pH 7.0).

pH Stability

The heat-resistant cysteine synthase of the present invention has excellent stability to high and low pH levels. It is preferable that the cysteine synthase of the invention exhibits activity under conditions in a range from about pH 6 to 10.

The heat-resistant cysteine synthase having the amino acid sequence set forth as SEQ ID NO: 2, that is one example of the invention, has an optimal pH of about 7.5 to 8.0.

The enzyme activity while estimating the pH stability is the value determined by the activity assaying method (3) described in the item of EXAMPLE.

Organic Solvant Resistance

The heat-resistant cysteine synthase of the present invention is resistant to organic solvents. It is preferable that the enzyme exhibits activity in buffer containing 20 vol % or more of an organic solvent such as for example ethanol, butanol, tetrahydrofuran, ethyl acetate and the like, for example. The maximum volume percentage of organic solvent in the buffer in which the cysteine synthase of the invention can exhibit enzyme activity is within the range in which the enzyme protein is not precipitated. Resistance to organic solvents is a characteristic feature of archaea.

Substrate Specificity

The cysteine synthase of the present invention is an enzyme which can catalyze the following scheme (I).

RH+CH₃COOCH₂—CHNH₂COOH→R—CH₂—CHNH₂COOH+CH₃COOH (I)

In the above scheme (I), R is SH⁻ or S₂O₃H⁻.

The enzyme of the present invention may be one permitting a reaction to be carried out in which S in the above reaction scheme (I) is Se instead.

The cysteine synthase of the present invention may also be an enzyme which can synthesize, from O-acetylserine and various nucleophiles, the corresponding amino acids. For example, the enzyme of the present invention may be an enzyme permitting cysteine to be synthesized from O-acetylserine and sodium sulfide or hydrogen sulfide. It may also be an enzyme permitting S-sulfocysteine to be synthesized from O-acetylserine and thiosulfuric acid, or an enzyme permitting β-pyrazolealanine to be synthesized from pyrazole and O-acetylserine. Examples of combinations of nucleophiles and products are given in Table 1 below. Any one of the cysteine synthase of the present invention need not necessarily enable all the reactions in Table 1 to be carried out, but the cysteine syntheses of the present Invention include those that allow any of the reactions in Table 1 to be carried out.

TABLE I

| Nucleophile | Product |
|---|---|
| Sulfide | 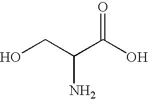 |
| Thiosulfuric acid | 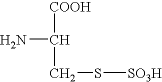 |
| Pyrazole | 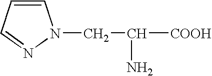 |
| Methanethiol | 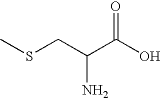 |
| Ethanethiol | 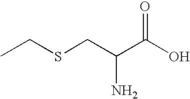 |
| Mercaptoethanol | 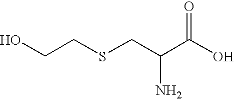 |
| Cysteine | 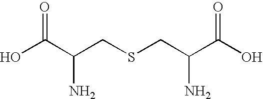 |
| Dithiothreitol | 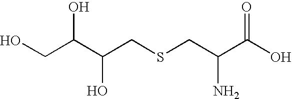 |
| Glutathione | 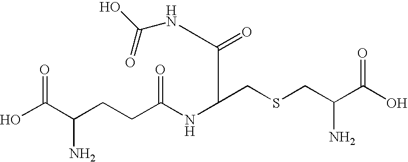 |
| Homocyateine | 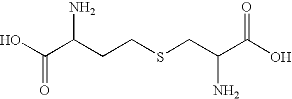 |
| 2-nitro-5-thiobenzoic acid | 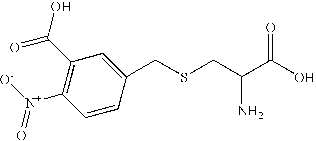 |
| Azide | 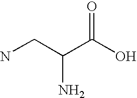 |
| Cyanide | 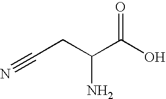 |
| Hydroxylamine | 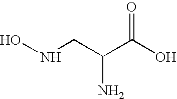 |

Because of its high heat resistance, the enzyme of the invention can act on highly concentrated substrate solutions prepared at higher temperatures than mesophilic or thermolabile cysteine syntheses and accordingly target compounds can be synthesized efficiently.

When a reaction is carried out using pyrazole, L-mimosine, or the like as substrate, because such substrates are poorly soluble in aqueous solution, the substrate solution must be prepared at elevated temperature and/or prepared with an aqueous solution in which an organic solvent has been dissolved to obtain a highly concentrated substrate solution. In these cases as well, the excellent heat resistance and organic solvent resistance of the cysteine synthase of the present invention will allow highly concentrated substrate solutions to be obtained, and thus more efficient synthetic reactions to be carried out.

Bacteria Producing the Enzyme

Examples of heat-resistant cysteine synthase of the present invention include those produced by hyperthermophilic microorganism, such as genuses Aeropyrum, Pyrococcus, Sufolobus, Thermoplasma, Thermoproteus, Mastigocladus, Bacillus, Synechococcus, and Thermus. That produced by the hyperthermophilic archaea Aeropyrum pernix is preferred.

Amino Acid Sequence

Examples of cysteine synthase of the present invention include polypeptides having the following amino acid sequences:

(1) a polypeptide comprising the amino acid sequence set forth in SEQ ID NO: 2.

(2) a polypeptide comprising an amino acid sequence containing one or more amino acid residue deletions, substitutions or additions to SEQ ID NO: 2 and has heat-resistant cysteine synthase activity; i.e., a polypeptide which has the amino acid sequence of SEQ ID NO: 2 with one or more amino acid residues deleted, substituted or added and exhibits heat-resistant cysteine synthase activity.

Polypeptide (2) Is preferably a polypeptide comprising the amino acid sequence of SEQ ID NO: 2 with about 1 to 50, and particularly about 1 to 25 amino acid residues deleted, substituted, or added, although up to 30% of the number of amino acid residues of SEQ ID NO: 2 may be modified in regions that are not conserved among cysteine syntheses.

Specifically, when substituting amino acids, an amino acid can for example be substituted with an amino acid having properties that are similar in terms of polarity, charge, solubility, hydrophilicity/hydrophobicity, and the like, in the interests of maintaining the structure of the protein. For example, glycine, alanine, valine, leucine, isoleucine, and proline are classified as nonpolar amino acids; serine, threonine, cysteine, methionine, asparagine, and glutamine are classified as polar amino acids; phenylalanine, tyrosine, and tryptophan are classified as amino acids with aromatic side chains; lysine, arginine, and histidine are classified as basic amino acids; and aspartic acid and glutamic acid are classified as acidic amino acids. Amino acids should therefore be substituted with amino acids of the same group.

Among polypeptides of (1), examples of polypeptides comprising the amino acid sequence set forth in SEQ ID NO: 2 include polypeptides having a length no greater than 3 times that of the amino acid sequence set forth in SEQ ID NO: 2. Similarly, among polypeptides of (2), examples of polypeptides comprising the amino acid sequence containing one or more amino acid residue deletions, substitutions, or additions to SEQ ID NO: 2 include polypeptides having a length no greater than 3 times that of the amino acid sequence containing one or more amino acid residue deletions, substitutions, or additions to SEQ ID No: 2.

The polypeptide consisting of the amino acid sequence In SEQ ID NO: 2 is preferred among the polypeptides of (1). The Polypeptide consisting of amino acid sequence containing one or more amino acids deleted, substituted, or added to SEQ ID NO: 2, and exhibiting heat-resistant cysteine synthase activity, is preferred among polypeptides of (2).

The enzyme of the present invention can be obtained by culturing bacteria which produce the enzyme, followed by recovering the enzyme from the liquid containing ruptured cells and further purifying the enzyme. It can also be obtained by chemical synthesis based on the amino acid sequence in SEQ ID NO: 1. It can also be obtained by the method of the invention described below.

(II) DNA of the Invention

The DNA of the present invention is the DNA of (3), (4), or (5) below:

(3) DNA comprising the base sequence set forth in SEQ ID NO: 1.

(4) DNA comprising DNA which hybridizes, under stringent conditions, with DNA consisting of the base sequence in SEQ ID NO: 1, and encodes for a polypeptide exhibiting heat-resistant cysteine synthase activity.

(5) DNA encoding the above polypeptide of the present invention.

In the present invention, “stringent conditions” includes conditions under which hybridization is brought about at 68° C. in ordinary hybridization solution, and conditions under which hybridization is brought about at 42° C. in a hybridization solution containing 50% formaldehyde. Specifically, examples include the conditions used for Southern hybridization noted in “Molecular Cloning: A Laboratory Manual”, 2^{nd} Ed., vol. 2.

The DNA of (4) is preferably DNA comprising DNA encoding the polypeptide consisting of the amino acid sequence of SEQ ID NO: 2, with about 1 to 50, and particularly about 1 to 25 amino acid residues deleted, substituted, or added. However, it may be DNA comprising DNA encoding a polypeptide in which up to 30% of the entire amino acid sequence In SEQ ID NO: 2 is modified in regions that are not conserved among cysteine syntheses.

Examples of DNA comprising the base sequence set forth in SEQ ID NO: 1 among DNA of (3) Include DNA having a length no greater than 3 times that of the base sequence set forth in SEQ ID NO: 1. Examples of DNA comprising DNA which hybridizes, under stringent conditions, with DNA consisting of the base sequence in SEQ ID NO: 1, and encodes a polypeptide exhibiting heat-resistant cysteine synthase activity, can include DNA having a length no greater than 3 times that of DNA which hybridizes, under stringent conditions, with DNA consisting of the base sequence in SEQ ID NO: 1 and encodes a polypeptide having heat-resistant cysteine synthase activity.

Among DNA of (3), DNA consisting of the base sequence set forth in SEQ ID NO: 1 is preferred. Among DNA of (4), DNA that hybridizes under stringent conditions with DNA consisting of the base sequence set forth in SEQ ID NO: 1 and encodes for a polypeptide having heat-resistant cysteine synthase activity is preferred.

The DNA of the present invention can be isolated from a chromosomal DNA library of thermophilic bacteria such as genuses Aeropyrum, Pyrococcus, Sufolobus, Thermoplasma, Thermoproteus, Mastigocladus, Bacillus, Synechococcus, and Thermus by hybridization using a probe. It can also be amplified by PCR based on the DNA sequence in SEQ ID NO: 1. It can also be obtained by chemical synthesis.

The aforementioned modified cysteine synthase DNA of (4) can be prepared by known methods such as chemical synthesis, genetic engineering, or mutagenesis. Examples of genetic engineering Include known methods such as the introduction of DNA deletions with exonucleases, the introduction of linkers, site-directed mutagenesis, and the modification of base sequences by PCR using variant primers on available cysteine synthase DNA.

(III) The Vector of the Invention

The vector of the invention is the recombinant vector comprising the DNA of the invention described above. A wide range of known vectors can be utilized as the vector to be integrated with the DNA of the invention. Vectors for bacteria, as well as vectors for yeasts and animal cells can be utilized. With regard to enzyme producing efficiency, vectors for bacteria may ordinarily be used. Examples of known vectors include E. coli vectors such as pBR322, pUC19, and pKK233-2, vectors for genus Bacillus such as pUB110, pC194, pH 194, pTHT15, and pBD16, vectors for yeasts such as Yip5, Yrp17, and Yep24, and vectors for animal cells such as pUC18, pUC19, and M13 mp18.

(IV) The Transformant of the Invention

The transformant of the invention is one comprising the recombinant vectors of the invention as described above. As host, bacterial cells, yeasts, animal cells, and the like can be used depending on the vector. Bacillus subtilis, Bacillus brevis, yeasts, fungi, and the like are preferred as host in view of mass production of the target protein.

Transformation can be brought about by known methods such as the calcium phosphate method, protoplast method, electroporation, spheroplast method, lithium acetate method, lipofection, and microinjection. The method may be selected from among such known methods according to the kind of host.

(V) Method for Producing Cysteine Synthase of the Invention

The method for producing the cysteine synthase of the present invention comprises incubating the transformant of the invention and recovering the cysteine synthase from the transformant.

The enzymes produced by the transformant can be purified by combinations of known methods of protein purification, such as centrifugation, salting out, precipitation by solvent, dialysis, ultrafiltration, gel filtration, ion exchange chromatography, affinity chromatography, reversed phase HPLC, etc.

Intracellularly or intraperiplasmically produced enzymes of the invention may be purified after the cells have been ruptured by known methods such as ultrasonic treatment or surfactant treatment.

When the heat resistant cysteine synthase of the present invention is purified, it is preferable that the purification step includes incubation of a solution of the protein to be purified, generally for about 10 to 120 minutes, and particularly about 10 to 30 minutes, at a temperature which usually results in 60% or more, and particularly 80% or more, residual activity after about 10 to 30 minutes (and particularly about 20 minutes) of incubation of the enzyme of the invention, and which is generally 10° C. or more, and particularly 15° C. or more, higher than the critical growth temperature of the host producing the enzyme. This allows protein impurities produced by the host to be denatured and inactivated, with virtually no loss of target enzyme activity. After the heat treatment step, the protein solution can be centrifuged for example but not limited to for about 20 minutes at about 15,000 rpm to allow the denatured protein impurities to be precipitated. This heat treatment step may be carried out at any stage of the purification process.

Such a heat treatment process can be carried out not only for the purification of the heat-resistant cysteine synthase of the invention, but for the purification of any heat resistant protein, thereby dramatically improving the purity of the heat resistant protein.

(VI) Method for Synthesizing Amino Acids

To synthesize a variety of amino acids using the cysteine synthase of the present invention, the enzyme may be added to a substrate solution comprising the substrate dissolved In buffer such as about 50 to 100 mM phosphate buffer or carbonate buffer (the pH being the optimal pH of the enzyme used), and incubated at the optimal temperature. The synthetic reaction may be carried out at a temperature where the enzyme activity can be maintained, and preferably at the optimal temperature. When the substrate is poorly soluble in aqueous solution, the temperature of the substrate solution may be raised and/or organic solvent may be added to the substrate solution within a range that will not result in very much decline in enzyme activity.

A coenzyme such as pyridoxal 5′-phosphate is usually used when such a synthesis is carried out using the enzyme of the invention. It is preferable that the coenzyme is used at a molar ratio of about 1000 to 10000-fold in excess based on the enzyme of the invention, and particularly about 1000 to 2500-fold.

### EXAMPLES

The present invention is illustrated in further detail in the following examples, but the invention is not limited to these examples.

### Example 1

### Cloning of Cysteine Synthase Gene of Aeropyrum pernix K1

i) Preparation of Chromosomal DNA

Medium was prepared by dissolving 37.4 g Bacto Marine medium (Difco) and 1.0 g of Na₂S₂O₃—5H₂O in 1 L water, and then adjusting the pH to between 7.0 and 7.2. Hyperthermophilic archaea Aeropyrum pernix K1 (registered as JCM9820 at The Institute of Physical and Chemical Research) was inoculated in the medium and incubated for 3 days at 90° C. with shaking. The bacteria were harvested by centrifuging the culture broth at 5,000 rpm for 10 minutes.

The bacterial cells were washed twice with 10 mM Tris (pH 7.5)-1 m EDTA solution, and then sealed in InCert agarose block (manufactured by FMC). The block was treated with 1% N-lauroylsarcosine-1 mg/mL protease K solution, thereby chromosomal DNA was isolated in the agarose block. The conditions under which the chromosomal DNA were isolated using the InCert agarose block were guided by the manual attached to the agarose block.

ii) Amplification of Cysteine Synthase Gene

DNA comprising the base sequence in SEQ ID NO: 1 was amplified by PCR. The PCR conditions were guided by the manual attached to the PCR kit. An oligonucleotide primer having the DNA sequence beginning from bass number 1 (that is, beginning from the initiation codon) in the DNA sequence set forth in SEQ ID NO: 1 of the sequence listing was synthesized as the primer corresponding to the 5′ end. A primer corresponding to the region downstream from the 3′ end of the base sequence in SEQ ID NO: 1 in the chromosomal DNA of Aeropyrum pernix K1 being a primer producing amplified DNA with cutting site by restriction enzyme BamHI, was synthesized as the primer corresponding to the 3′ end. After the PCR reaction, the amplified DNA was completely digested (for 3 hours at 37° C.) with restriction enzyme BamHI. The cysteine synthase gene was then purified using a purification column kit.

iii) Construction of Vector Containing Cysteine Synthase Gene

The pET-8c vector (manufactured by Novagen) was cleaved with restriction enzyme NcoI and purified, and the ends were then blunted using T4 DNA polymerase. The purified plasmid was cleaved and purified with restriction enzyme BamHI. The pET-8c plasmid cleaved with BamHI was then ligated with the aforementioned cysteine synthase gene cleaved with BamHI by reaction with T4 ligase at 16° C. for 16 hours. The ligated DNA was used to transform competent cells of E. coli XL2-BlueRF′ (manufactured by Novagen). Transformants were selected on the basis of colony formation on LB agar plates containing 0.05 mg/mL ampicillin. Plasmids containing the cysteine synthase gene were extracted from the transformants by the alkaline lysis method.

### Example 2

### Production of Cysteine Synthase

i) Preparation of Transformants Containing the Cysteine Synthase Gene

To 1.5 mL tubes were added 0.04 mL (2,000,0000 cfu/μg) of competent cells of E. coli Rosetta (DE3) (manufactured by Novagen) and 0.003 mL of DNA solution (8.4 ng plasmid DNA) of plasmids containing the cysteine synthase gene prepared above. The tubes were left to stand for 30 minutes in ice, and given heat shock treatment of 42° C. for 30 seconds. To the tubes, 0.25 mL SOC medium was then added, and the bacteria were incubated for 1 hour at 37° C. with shaking. The culture broth was then applied to LB agar plates containing ampicillin and chloramphenicol, incubated overnight at 37° C., thereby obtaining transformants.

ii) Purification of Cysteine Synthase

The resulting transformants were inoculated in NZCYM medium containing ampicillin and chloramphenicol, and incubated at 37° C. until the absorbance of the bacterial culture at 600 nm reached 0.5, IPTG (isopropyl-β-D-thiogalactopyranoside) was then added to increase the expression of the plasmid, and the transformants were incubated for another 19 hours. The culture broth was centrifuged at 8,000 rpm for 10 minutes to harvest the bacterial cells.

To 2.8 g of the harvested bacterial cells, 50 mL of 50 mM potassium phosphate buffer (pH 7.6) containing 1 MM DTT, 1 mM EDTA, 0.2 mM pyridoxal 5′-phosphate and 0.15 M NaCl was added, and the cells were ultrasonically ruptured for 3 minutes at an output power of 90 W. The ruptured cells were centrifuged at 15,000 rpm for 30 minutes, and the supernatant was collected.

To eliminate protein contaminants by precipitation, the supernatant was heated for 20 minutes at 85° C., centrifuged at 15,000 rpm for 20 minutes, and the supernatant was collected. Solid ammonium sulfate was added to the supernatant to give a concentration based on the resulting solution of 50%, the obtained protein precipitate was collected in a centrifuge, dialyzed against 50 mM potassium phosphate buffer (pH 7.6) containing 1 mM DTT, 2 mM EDTA and 0.2 mM pyridoxal 5′-phosphate, and applied to a Hitrap Q (manufactured by Pharmacia) column of anion exchange resin equilibrated with the same buffer. The active fractions were dialyzed against 50 mM potassium phosphate buffer (pH 7.6) containing 1 mM DTT, 1 mM EDTA, 0.2 mM pyridoxal 5′-phosphate, and 0.15 M NaCl, and equilibrated with the same buffer. Gel filtration chromatography was performed using a gel filtration Superdex 200 (Pharmacia) column. The resulting active fractions contained a homogenous preparation giving a single band by SDS-PAGE.

Gel filtration chromatography revealed the enzyme to have a molecular weight of about 65 kDa.

iii) Assaying Method of Cysteine Synthase (O-acetylserine Sulfhydrylase) Activity

The cysteine synthase was detected in the purification step by assaying the cysteine synthesis activity or S-sulfocysteine synthesis activity in the following manner:

\mathring{1} Cysteine Synthesis Activity

To 0.3 mL of 100 mM potassium phosphate buffer (pH 7.5) containing 1 mM EDTA, 0.2 mM pyridoxal 5′-phosphate, 20 mM O-acetyl-L-serine as substrate, and 2 mM sodium sulfide (Na₂S) as substrate, is added 0.8 μg enzyme solution and the reaction is carried out for about 4 minutes at 40° C. To stop the reaction, 0.15 mL of 20% trichloroacetic acid solution is added to the reaction solution, and the absorbance at 560 nm is measured by ninhydrin reaction to quantify the amount of cysteine produced.

\mathring{2} S-Sulfocysteine Synthesis Activity

The same procedure as method \mathring{1} above is conducted except that 20 mM thiosulfuric acid is used instead of the 2 mM sodium sulfide and 3 μg purified enzymes is added to the substrate solution. A reaction is brought about for 4 minutes at 40° C. To stop the reaction, 0.15 mL of 20% trichloroacetic acid solution is added to the reaction solution, the reaction solution is centrifuged for 3 minutes at 15,000 rpm because precipitate is produced, and the supernatant is recovered. The absorbance of the supernatant at 560 nm is measured by the ninhydrin method to quantify the amount of S-sulfocysteine produced.

### Example 3

### Identification of Cysteine Synthase Gene from Hyperthermophilic Archaea Aeropyrum pernix K1

SEQ ID NO: 1 shows the DNA sequence of the cysteine synthase gene in the chromosomal DNA of the hyperthermophilic archaea Aeropyrum pernix K1 obtained in Example 1. SEQ ID NO: 2 gives the amino acid sequence of the cysteine synthase of the hyperthermophilic archaea Aeropyrum pernix K1 obtained in Example 2.

### Example 4

### Properties of Heat-Resistant Cysteine Synthase

The properties of the cysteine synthase from the Aeropyrum pernix K1 obtained by the above method were evaluated.

i) Optimal pH

The optimal pH was determined by the following assaying method (3) for the cysteine synthase activity.

Assaying Method of Cysteine Synthase Activity (3)

To 0.3 mL of buffer containing 20 mM O-acetylserine and 2 mM sodium sulfide as substrates, 1 mM EDTA and 0.2 mM pyridoxal 5′-phosphate, was added 0.8 μg purified enzyme. A reaction was brought about for 4 minutes at 40° C., and the amount of cysteine synthesized was determined by the ninhydrin method to assay the enzyme activity. 50 mM potassium phosphate buffer (pH 6.2 to 7.8) and 50 mM sodium carbonate (pH 7.6 to 9.9) were used as buffers.

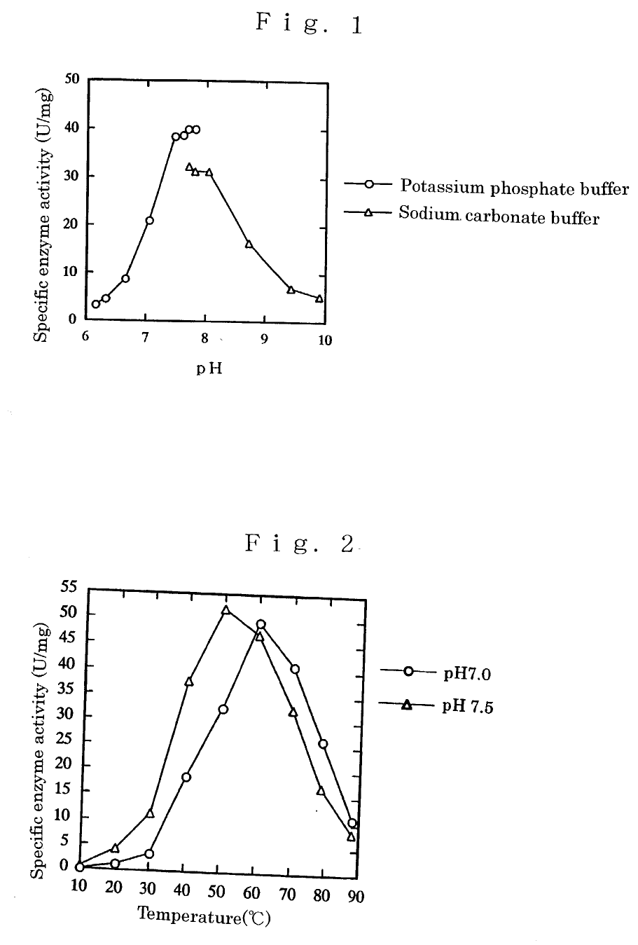

As the results in FIG. 1 show, the optimal pH of the enzyme was 7.5 to 8.0.

ii) Optimal Temperature

The optimal temperature was determined by the following assaying method (2) for cysteine synthase activity.

Assaying Method of Cysteine Synthase Activity (2)

Purified enzyme was added to 50 mM potassium phosphate buffer (pH 7.5 or 7.0) containing 20 mM O-acetylserine and 2 mM sodium sulfide as substrates, and 0.2 mM pyridoxal 5′-phosphate so as to establish an enzyme concentration of 0.8 μg/mL, the reaction was brought about for 4 minutes, and the amount of cysteine synthesized was determined by the ninhydrin method to assay the enzyme activity.

As the results in FIG. 2 show, the optimal temperature was about 50° C. when pH 7.5 buffer was used, and was about 60° C. when pH 7.0 buffer was used. In the specific activity of the enzyme given in FIG. 2, 1 U is the amount of enzyme producing 1 μmol cysteine per minute.

iii) Heat Resistance

The optimal pH was determined by the following assaying method (1) for cysteine synthase activity.

Assaying Method of Cysteine Synthase Activity (1)

Four enzyme solution samples were prepared by adding cysteine synthase to 50 mM potassium phosphate buffer (pH 6.7) containing 1 mM EDTA and 0.2 mM pyridoxal 5′-phosphate so as to obtain an enzyme concentration of 0.3 mg/mL. The samples were incubated for 6 hours at 4° C., 80° C., and 100° C., respectively, to assay residual activity.

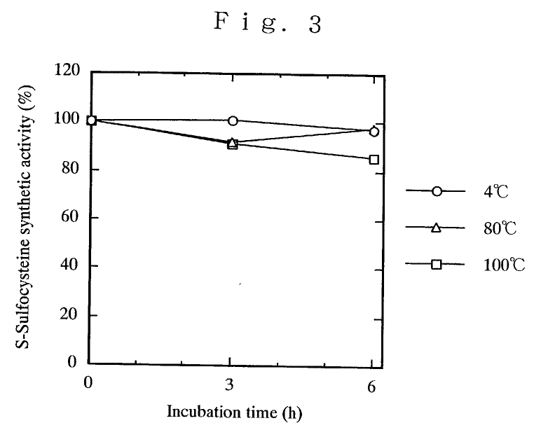

The results are given in FIG. 3. The enzyme had about 97% residual activity after 6 hours of incubation at 80° C., and about 85% residual activity after 6 hours of incubation at 100° C.

1. Heat-resistant cysteine synthase of which enzymatic activity is substantially unimpaired by heat treatment at 80° C. for 6 hours.

2. Heat-resistant cysteine synthase according to claim 1, derived from hyperthermophilic archaea Aeropyrum pernix.

3. Heat-resistant cysteine synthase with at least 80% residual activity after heat treatment at 100° C. for 6 hours.

4. Heat-resistant cysteine synthase according to claim 3, derived from hyperthermophilic archaea Aeropyrum pernix.

5. A polypeptide of (1) or (2) below:
(1) a polypeptide comprising the amino acid sequence set forth in SEQ ID No: 2.
(2) a polypeptide which comprises the amino acid sequence in SEQ ID NO: 2 but containing one or more deletions, substitutions or additions of amino acid residues and has heat resistant cysteine synthase activity.

6. DNA of (3), (4), or (5) below:
(3) DNA comprising the base sequence set forth in SEQ ID NO: 1.
(4) DNA comprising DNA which hybrides, under stringent conditions, with DNA consisting of the base sequence in SEQ ID NO: 1, and encodes a polypeptide having heat-resistant cysteine synthase activity.
(5) DNA encoding the polypeptide according to claim 5.

7. A vector comprising the DNA according to claim 6.

8. A transformant comprising the vector according to claim 7.

9. A method for producing heat-resistant cysteine synthase which comprises incubating the transformant according to claim 8, and recovering the cysteine synthase from the transformant.
